(19) United States
(12) Patent Application Publication
(10) Pub. No.: US 2022/0387180 A1
(43) Pub. Date: Dec. 8, 2022

(54) ORTHOPEDIC IMPLANT

(71) Applicant: KUMOVIS GMBH, München (DE)

(72) Inventors: Stefan LEONHARDT, Munich (DE); Sebastian PAMMER, Munich (DE)

(21) Appl. No.: 17/755,953

(22) PCT Filed: Nov. 16, 2020
(86) PCT No.: PCT/EP2020/082305

§ 371 Date: May 12, 2022

(30) Foreign Application Priority Data
Nov. 15, 2019 (DE) 20 2019 106 375.0

(51) Int. Cl. A61F 2/30

(52) U.S. Cl. CPC A61F 2/30942; A61F 2002/30062; A61F 2002/30985; A61F 2002/3093; A61F 2002/30948; A61F 2002/30971; A61F 2002/30065

(57) ABSTRACT

The present disclosure relates to an orthopedic implant, wherein the implant is a 3D printed part and comprises at least one first portion and at least one second portion, the first portion forming a support structure and the second portion being at least partially made of a biodegradable material.

The present disclosure further relates to a method of manufacturing an orthopedic implant.

### TECHNICAL FIELD

The present disclosure relates to an orthopedic implant, the implant being a 3D printed part.

### BACKGROUND AND SUMMARY

Orthopedic implants produced by 3D printing are already known from the prior art.

For example, DE 10 2006 029 298 A1 discloses a material system for 3D printing. Here, the opportunity of manufacturing implants by so-called additive manufacturing is provided in the form of a dispersion for the manufacturing of granules by spray or fluid bed granulation.

EP 3 172 037 A1 relates to a method of manufacturing a component of at least one additive manufacturing process, the component being manufactured as a whole or in part from liquid raw material. Here, too, 3D printing makes it possible to manufacture implants.

Furthermore, a so-called gingiva former is known from DE 10 2014 105 884 A1; also in this case, corresponding adaptations of the implant to the actual conditions in the body are utilized by means of 3D printing and additive manufacturing.

It is the object of the present disclosure to further develop an implant of the aforementioned type in an advantageous manner, in particular to the effect that a load-bearing implant can be manufactured by means of 3D printing, which at the same time exhibits improved biocompatibility and tolerability.

This object is achieved according to the disclosure by an orthopedic implant. According to this, provision is made that an orthopedic implant is provided, the implant being a 3D printed part and comprising at least one first portion and at least one second portion, the first portion forming a support structure and the second portion being at least partially made of a biodegradable material.

The disclosure is based on the basic concept that—similar to the structure of a bone—a first, for instance outer, portion is made available, which provides a certain stiffness and mechanical strength for the implant, whereas a second, for instance inner, portion shall allow improved ingrowth. It is conceivable that the first portion encompasses or embraces the inner portion at least in parts. However, it is desirable that the second portion remains accessible so that tissue can grow into the inner portion. Complete manufacturing by means of 3D printing of the implant allows a very cost-effective manufacturing. In addition, it is made possible, for instance, to address individual anatomical conditions and requirements and to individually customize the shape of the implant.

It is conceivable that the implant is printed on the basis of patient data such as computer tomography image data or comparable data sets that have been obtained by means of imaging methods (such as magnetic resonance imaging, X-ray, fluoroscopy, etc.).

Here, for instance, provision can be made that the 3D printer has an interface via which the image data are imported. The 3D printer may be set up so as to translate the image data into print data in semi-automatic or automatic fashion and then to start the printing process accordingly (automatically).

In particular, provision can be made that the first, for instance outer, portion is at least partially made of polyether ether ketone (PEEK). PEEK is a high-strength plastic material (compared with other plastics) and at the same time a high-temperature-resistant thermoplastic material which, in addition to properties in terms of biocompatibility, can also provide the necessary strength for bone implants or bone replacement implants. Furthermore, it is also conceivable to use other high-performance plastics such as polyether ketone ketones (PEKK), polyphenylene sulfone (PPSU), polyaryl ether ketones (PAEK), polyethylene imine or polyether imides (PEI) or polyamide imide (PAI).

Furthermore, it may be provided that the second, for instance inner, portion is at least partially made of a biodegradable material, wherein the biodegradable material is one of the following materials or material combinations: polydioxanone (PDS or PPDX or PPDO), polylactide (PLA), poly(lactide-co-glycolide) (PLGA), a mixture of polylactide (PLA) and poly(lactide-co-glycolide) (PLGA).

Poly-p-dioxanone (poly-1,4-dioxasn-2-one)—often abbreviated as PDS, PPDX or PPDO—is a poly(ether-ester), which is virtually an alternating copolymer of ethylene glycol and glycolic acid and is made by ring-opening polymerization from 1,4-dioxan-2-one. In addition to homopolymers, a number of random copolymers and block copolymers, mostly with other lactone monomers such as glycolide, lactide or ε-caprolactone have been described. Poly-1,4-dioxan-2-one was introduced under the name PDSTM (polydioxanone sutures) in the form of monofilaments as the first absorbable, i.e., biodegradable, surgical suture material in 1981.

Polylactides, colloquially also called polylactic acids (PLA for short), are synthetic polymers that belong to the polyesters. They are synthesized from many lactic acid molecules chemically bonded together.

Poly(lactide-co-glycolide) (PLGA) is a copolymer of the monomers lactide and glycolide, which can be used in various ratios. A polyester of D,L-lactic acid and glycolic acid is formed, which can be easily degraded by the human body. PLGA is used as a surgical suture material (Vicryl).

The above-mentioned materials have proven their worth in connection with absorbable materials. On the one hand, they are highly biocompatible and, on the other hand, they are of such nature that they allow good ingrowth of surrounding tissue.

It is also conceivable that the biodegradable material is also mixed with fillers. In this context, it is conceivable that biocompatible materials can be used as fillers, for example ceramics such as hydroxylapatite.

It is also conceivable that pharmacologically active substances are incorporated in the implant. It is conceivable that these substances are incorporated in particular in the biodegradable material. This can improve and support the process of implant ingrowth. It would be possible to apply growth factors, anti-inflammatories, analgesics or the like as medications, i.e. pharmacologically active substances.

In addition, provision can be made that the orthopedic implant has its first portion and/or its second portion formed to be porous at least in parts. This facilitates and allows improved ingrowth of the implant at the implantation site.

In particular, provision can be made that the implant is a so-called 2-component cage, in particular a 2-component backbone cage.

Furthermore, the present disclosure relates to a method of manufacturing an orthopedic implant.

According to this, provision is made that the method comprises at least the following steps:

a first portion of the implant is produced by means of 3D printing, in particular by means of the FLM method, the first portion forming a support structure of the implant;

a second portion is produced by means of 3D printing, in particular by means of the FLM method, the second portion being at least partially made of a biodegradable material.

According to the disclosure, the concept of the method is to manufacture all components of the implant using a 3D printing process.

Here, it is conceivable in particular that the 3D printing process is a so-called FLM method, i.e. a fused layer modeling method.

Fused Layer Modeling (FLM)/Fused Deposition Modeling is an additive method in which a supplied plastic wire (filament) is melted in a nozzle head. The emerging thin melt strand is then used to build up the contour and the filling of the desired geometry layer by layer. By using a removable support material in the area of overhangs, even complex geometries with cavities, inner structures and large wall thickness variations are possible here.

The manufacturing, in particular exclusive and complete manufacturing in a 3D printing process, allows a very cost-effective manufacturing of the implant. In addition, it is possible, for instance, to thus adapt the implant to the individual anatomical conditions and needs of the patient during manufacturing and to individualize the shape of the implant during manufacturing.

It is conceivable that the implant is printed by the 3D printer on the basis of patient data such as computer tomography image data or comparable data sets that have been obtained by means of imaging methods (such as magnetic resonance imaging, X-ray, fluoroscopy, etc.).

For example, provision can be made that the image data is imported into the 3D printer via an interface. The 3D printer then converts the image data into print data. This can be done semi-automatically or automatically by the 3D printer. The printing process can then be started accordingly (automatically).

The 3D printer may be a printer that is designed as in WO2019/068581A1. By printing in one printing process, it is possible to avoid contaminations and to print and manufacture (almost) under clean room conditions or, if the printer is in a corresponding environment, under clean room conditions or ultra clean room conditions.

Furthermore, provision can be made that the first portion and the second portion are joined by means of 3D printing. In particular, this results in the possibility of completely manufacturing the implant in one device or one 3D printer. By joining the components of the implant by means of 3D printing, it can be ensured that the components (where desired and necessary) are joined seamlessly and/or (are joined) even without further joining agents such as adhesives and the implant is manufactured as a whole. In addition, the dimensional stability and accuracy can be positively influenced because the portion printed first can be kept at temperature after its manufacturing and then the two portions can cool down together.

Furthermore, it is conceivable that the second portion is printed or imprinted onto or into the first portion. By generating the support structure in, for example, the first printing step or in a printing step preceding the printing of the second portion, it can be achieved that the second portion is generated with a precise fit and, for example, can be inserted into the first portion.

Furthermore, it can be alternatively provided that the first portion and the second portion are printed separately and are not joined by means of a printing method.

In particular, it is possible that the orthopedic implant is an orthopedic implant as described above or in the following exemplary embodiments.

### BRIEF DESCRIPTION OF THE FIGURES

Further advantages and details of the disclosure will now be explained with reference to an exemplary embodiment shown in more detail in the drawings in which:

FIG. 1 shows in a schematic view a cross-sectional drawing through a first exemplary embodiment of an orthopedic implant according to the disclosure;

FIG. 2 shows a further exemplary embodiment of an orthopedic implant according to the disclosure;

FIG. 3 shows a further exemplary embodiment of an orthopedic implant according to the disclosure; and

FIG. 4 shows a further exemplary embodiment of an orthopedic implant according to the disclosure.

### DETAILED DESCRIPTION

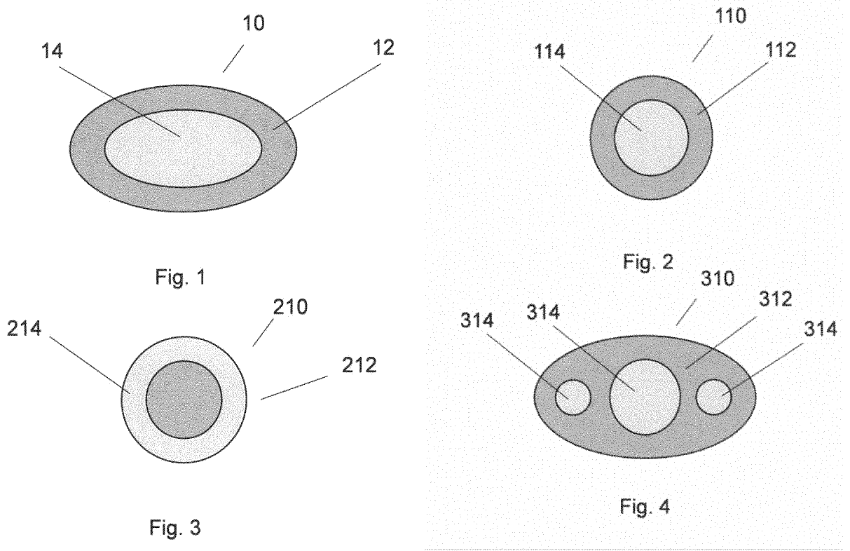

FIG. 1 shows an exemplary embodiment, according to the disclosure, of an orthopedic implant 10.

Here, the orthopedic implant 10 is a 3D printed part.

In addition, the implant 10 has a first, outer portion 12 and a second, inner portion 14.

The first, outer portion 12 is designed as a support structure in the form of a hollow bone type element.

The second, inner portion 14 completely fills the cavity and is entirely made of biodegradable material.

The first, outer portion 12 consists entirely of PEEK.

It is conceivable that fiber-reinforced PEEK is also used.

The second, inner portion 14 is made of a biodegradable material here, namely polydioxanone (PDS).

In principle, however, it is conceivable that PLA, PLGA or a mixture of PLA and PLGA is used. However, it is also conceivable to use any other absorbable and printable synthetic material.

The inner portion 14 is of porous design.

The implant 10 is a so-called 2-component cage.

FIG. 2 shows a further exemplary embodiment of the present disclosure.

The exemplary embodiment according to FIG. 2 also relates to an orthopedic implant 110.

The orthopedic implant 110 has all structural and functional features as the implant 10 according to FIG. 1.

Identical features or comparable features are designated with the identical reference sign, but increased by the value 100.

In contrast to the orthopedic implant 10 according to FIG. 1, the orthopedic implant 110 is basically of identical structure, but has a completely round cross-section. A design of this type is useful in particular in connection with implant pieces for the replacement of tubular bones or the like.

FIG. 3 shows a further exemplary embodiment of an orthopedic implant according to the present disclosure.

Here, the orthopedic implant 210 is also structured in the same way as the orthopedic implant 10 according to FIG. 1, but the following differences exist.

Identical or comparable features, however, are designated by the same reference sign, but increased by the value 200.

Here, the first portion 212 is arranged in the interior of the implant 210. The second portion 214 is arranged in such a way that it completely surrounds the inner, first portion 212 on the outside.

Here too, the cross-section is circular or nearly circular.

FIG. 4 shows a further exemplary embodiment of an orthopedic implant 310 according to the disclosure.

The orthopedic implant 310 also has all the features of the orthopedic implant 10. Identical or comparable features are designated with the same reference sign, but increased by the value 300.

Here, the first portion 312 has several cavities in which multiple second portions 314 are provided.

The first portion 312 is of oval design.

The latter accommodates three second portions 314 with biodegradable material, wherein two smaller circular second portions 314 are provided at the oval's ends and a larger portion 314 with a circular cross-section is provided in the middle.

In principle, it is envisaged that the implant 10, 110, 210, 310 can be printed or manufactured as described above in 3D printing using patient data.

A first filament, e.g. a PEEK filament, is used for the first portion.

For the second portion, a filament of a biodegradable material is used. The use of any biodegradable materials is conceivable, in particular the use of PDS, PLA, PLGA or mixtures thereof.

The method according to the disclosure for manufacturing an orthopedic implant, which may be an implant 10, 110, 210, 310 as described above, can be described as follows:

First, a first portion of the implant 10, 110, 210, 310 is produced by means of 3D printing.

In this process, the first portion 12, 112, 212, 312 forms a/the support structure of the implant 10, 110, 210, 310.

In a further step, a second portion 14, 114, 214, 314 is then produced by means of 3D printing.

The second portion 14, 114, 214, 314 is at least partially made of biodegradable material.

In particular, the first portion 12, 112, 212, 312 and the second portion 14, 114, 214, 314 are joined by means of 3D printing.

For this purpose, in one possible embodiment of the method, the second portion 14, 114, 214, 314 is printed onto the first portion 12, 112, 212, 312.

As an alternative, provision can be made that the first portion 12, 112, 212, 312 and the second portion 14, 114, 214, 314 are printed separately and are not joined by means of a printing method.

### REFERENCE SIGNS

10 orthopedic implant
12 first, outer portion
14 second, inner portion
110 orthopedic implant
112 first, outer portion
114 second, inner portion
210 orthopedic implant
212 first portion
214 second portion
310 orthopedic implant
312 first, outer portion
314 second, inner portion

1. An orthopedic implant, wherein the implant is a 3D printed part and comprises at least one first portion and at least one second portion, the first portion forming a support structure and the second portion being at least partially made of a biodegradable material.

2. The orthopedic implant according to claim 1, wherein the first, for instance outer, portion is at least partially made of PEEK and/or PEKK and/or PAEK and/or PEI and/or PPSU and/or PSU and/or PAI.

3. The orthopedic implant according to claim 1, wherein the second, for instance inner, portion is at least partially made of a biodegradable material, the biodegradable material being one of the following materials or material combinations: polydioxanone, polylactide, poly, a mixture of polylactide and poly.

4. The orthopedic implant according to claim 1, wherein the first portion and/or the second portion is/are formed to be porous at least in parts.

5. The orthopedic implant according to claim 1, wherein the implant is a 2-component cage.

6. A method of manufacturing an orthopedic implant, comprising at least the following steps:
a first portion of the implant is produced by means of 3D printing, wherein the first portion forms a support structure of the implant;
a second portion is produced by means of 3D printing, wherein the second portion is at least partially made of a biodegradable material.

7. The method according to claim 6, wherein the first portion and the second portion are joined by means of 3D printing.

8. The method according to claim 6, wherein the second portion is printed onto the first portion.

9. The method according to claim 6, wherein the first portion and the second portion are printed separately and are not joined by means of a printing method.

10. The method according to claim 6, wherein the orthopedic implant is an orthopedic implant.

11. The orthopedic implant according to claim 5, wherein the 2-component cage is a 2-component backbone cage.

12. The method of manufacturing an orthopedic implant according to claim 6, wherein the implant is produced by means of the FLM method.
